(19) United States
(12) Patent Application Publication
(10) Pub. No.: US 2006/0167375 A1
(43) Pub. Date: Jul. 27, 2006

(54) Skin testing kit for diagnosing atopy

(76) Inventors: Gaetan Terrassse, Montceau (FR); Yves Trehin, Toulouse (FR); Emile Loria, La Jolla, CA (US)

Correspondence Address: SHIHONG NICOLAOU, 4931 ARROYO LINDO AVE., SAN DIEGO, CA, 92117, US

(21) Appl. No.: 10/530,832

(22) PCT Filed: Oct. 8, 2003
(86) PCT No.: PCT/FR03/02957

§ 371 Date: Nov. 3, 2005

(30) Foreign Application Priority Data
Oct. 8, 2002 (FR) 02/12483

(51) Int. Cl. A61B 5/00

(52) U.S. Cl. 600/556

(57) ABSTRACT

The instrument for the allergies' detection is a cutaneous kit which has a main body, with a sharp edge (5) around its circumference at the lower surface (4). At least one cavity (21) or hollow space is coaxial to the body (1) for a multi-point needle (8). The outer circumference of the body is bonded to the inner edge of a flexible ring (16), in turn held by a rigid support (17) at its outer edge. The whole of the lower surface of the kit is shrouded by a blister. The cavity and the surrounding space (14) is filled with at least one allergenic substance. The instrument can contain three bodies with their needle assemblies.

The present invention concerns the tests for the allergies' detections, in adults and/or children.

With 20% of the European population suffering from allergies and an equivalent number of allergic people in other industrialized countries, the allergic diseases represent today a true problem of public health. Accordingly, WHO ranks allergic diseases the fourth problem of health. In such a context, it is of primary importance to give to the medical profession the means of detecting as soon as possible the atopic subjects, especially the children.

Prior art already comprises many tests aiming at detecting the allergic diseases. The American patent U.S. Pat. No. 5,075,077 described an automat allowing to detect specific IgE in biological samples submitted to various allergens. This biological test comprises several disadvantages: it requires a blood sample, which can be an obstacle to the early diagnosis of allergy in particular in infants, and the predictive value of this biological test is only 55% (study Matricardi P M, Nizini R, Pizzolo J G, D'Angelio R. Use of Phadiatop in mass-screening programs of inhaling allergy: advantage and limitation. Clin Exp Allergy 1990; 20: 151-155).

The U.S. Pat. No. 5,104,620 described a kit of cutaneous tracking of the allergy including a base provided with multiple chambers containing allergens compositions. An upper plate comprises push-buttons provided with a needle on their lower part, each push-button being placed opposite to the chamber, and being provided on its lower face with a needle and on its upper face with a dome. A pressure could be applied to the push-button, which will cause a lowering of the needle, the perforation of the base of the chamber and the penetration of the point covered with allergen composition in the patient's skin.

Patent WO 80/00531 described a kit of tracking the allergy including a base provided with recessions and a well containing an allergen composition and intended to receive a scarification's point bathing in the allergen composition. By pressure on the sleeve surrounding the base of the point, the scarification's point will go down, perforate the well as well as the base, and will penetrate into the patient's skin.

However, no prior art describes a cutaneous kit which at the same time ensures a reliable and constant exposure, at cutaneous and sub-cutaneous levels, to several allergens in only one medical gesture, and allows the delimitation of the exposure to the allergen, and a reliable identification of the allergic reactions when reading the test.

The purpose of the present invention is to propose a cutaneous diagnostic kit for the patient's atopy which can be applied by a doctor or a nurse, characterized in that it includes:

at least a central body of cylindrical external form of which the upper face is plane and bonded to a single body of gripping and pressure and whose lower face presents a cutting sparing making an acute edge on the external circumference of the aforesaid central body,

at least a cavity or coaxial hollow little cell of the body central, this cavity or little cell being part or bonded to the central body, having a height such as its lower end exceeds the horizontal plane of the acute end of the edge, and supporting at this lower end a multi-points needle, the central body being bonded on all its circumference to the inner edge of a flexible ring, the outer edge of the flexible ring being bonded on all its circumference to a rigid support,

a blister fixed on the lower face of the support and covering the totality of the lower surface of the kit,

at least an allergen composition, located in the cavity or the little cell as well as in the existing space between the blister and the lower faces of the flexible ring and of the central body.

According to a mode of realization of the invention, the kit comprises three central bodies. A preferred mode of realization of the invention expects that the distance separating the first and the second central body is different from the distance separating the second and the third central body, each distance being different from the distance separating the first and the third central body.

Preferably, the distance between the first and the second central body lies between 1 centimeter and 3 centimeters, the distance between the second and the third central body lies between 2 centimeters and 4 centimeters and the distance between the second and the third central body lies between 3 centimeters and 7 centimeters.

According to an alternative of the invention, the cavity or the little cell has a height ranging between 0.2 centimeter and 1 centimeter.

According to a mode of realization of the invention, the multi-points needle presents at least a crown on which the points are fixed or positioned.

A preferred mode of realization of the invention provides that the multi-points needle presents several concentric crowns.

According to a particular mode of realization of the invention, the multi-points needle contains 8 points equally distributed, the aforementioned 8 points being distributed on one or two crowns.

Preferably, the multi-points needle presents two concentric crowns, each crown presenting 4 points angularly positioned in alternation.

According to a mode of realization of the invention, the allergen composition can be in gel form, in liquid form, in the form of emulsion or a suspension.

A particular mode of realization of the invention provides that the allergen composition includes a mixture of pollens' allergens.

According to a preferred mode of the invention, the mixture of pollens' allergens includes graminaceous pollens, herbaceous pollens and trees' pollens.

Preferably, the mixture of pollens' allergens includes a high peptide concentration of proteins PR10, pectase lyase and profilines, natural or synthetic.

A particular mode of realization of the invention provides that the allergen composition includes a mixture of food allergens.

Preferably, the mixture of food allergens includes a high peptide concentration of chitinases, Lipids Proteins Transfer, natural or synthetic.

A particular mode of realization of the invention provides that the allergen composition includes a mixture of domestic environmental allergens.

According to a preferred mode of the invention, the mixture of domestic environmental allergens includes acarids' allergens, superficial body growths of dog and cat, as well as moulds.

Preferably, the mixture of domestic environmental allergens includes a high peptide concentration of tropomyosines, profilines and cystine proteases, natural or synthetic.

A preferred mode of realization of the invention provides that each central body contains in its space an allergen composition different from that of the other central bodies.

A mode of realization preferred of the invention provides that the kit consists of materials for single use.

One will understand better the invention with the description, made hereafter on a purely explanatory basis, of a mode of realization of the invention, in reference to the annexed figures:

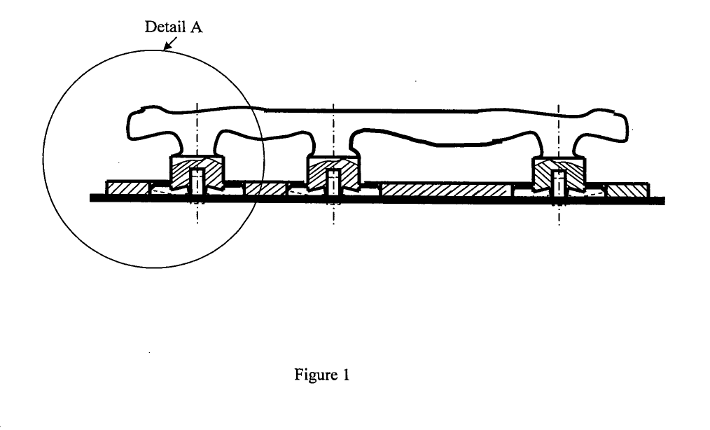

FIG. 1 is a vertical cross-section of the device according to the invention;

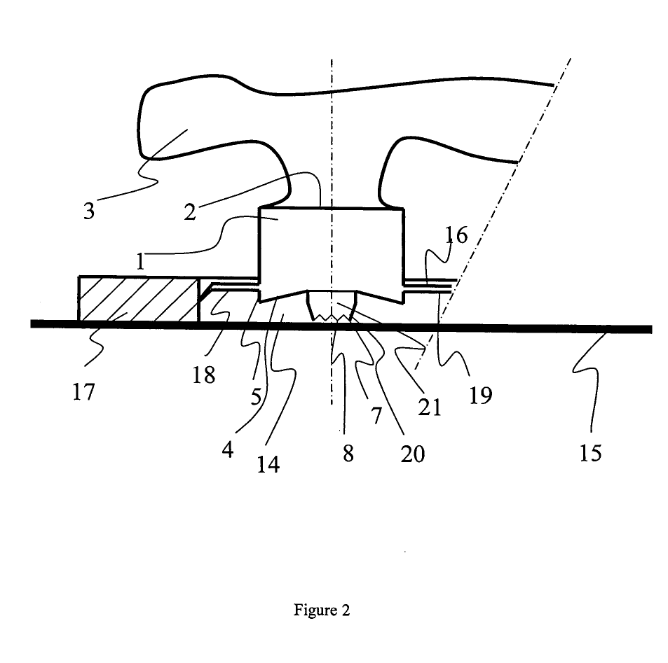

FIG. 2 is a vertical cross-section of a mode of realization of the detail; surrounded on FIG. 1, according to which interior cutting 20 defines a cavity 21;

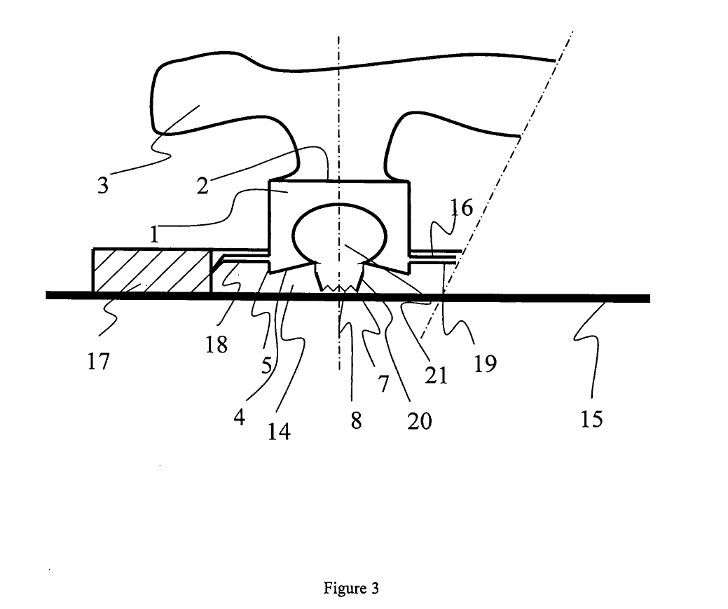

FIG. 3 is a vertical cross-section of another mode of realization of the detail surrounded on FIG. 1, according to which interior cutting 20 defines a cavity 21 which is prolonged inside the central body 1;

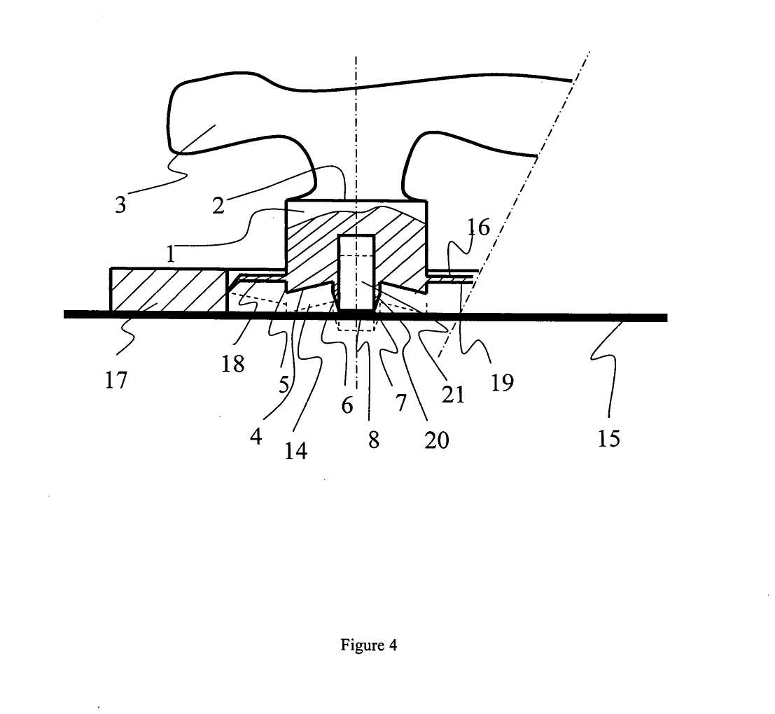

FIG. 4 is a vertical cross-section of another mode of realization of the detail surrounded on FIG. 1, in which cavity 21 and interior cutting 20 define a little cell 6;

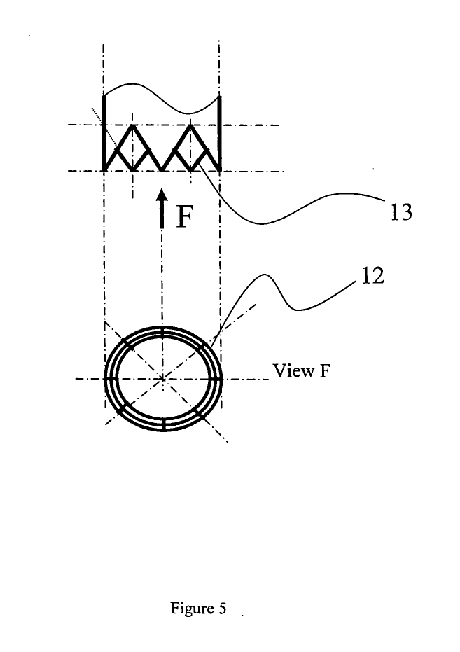

FIG. 5 is a detail of the multi-points needle;

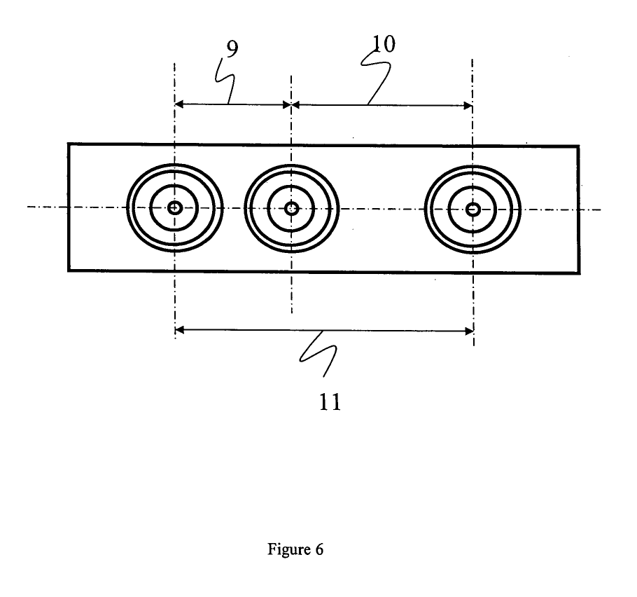

FIG. 6 is a view of the lower part of the device.

The purpose of the cutaneous kit according to the invention is a simple and fast diagnosis of the patient's atopy applied by a doctor or a nurse. This easiness of use also lies into the fact that the kit is for single use and is manufactured with disposable materials.

FIG. 1 represents a mode of realization of the device according to the invention, which includes three central bodies 1. The central bodies 1 are preferably of cylindrical external form, but other forms can be considered, in particular cubic or parallelepipedic ones. Each central body 1 comprises a upper face 2 and one lower face 4. The upper face 2 of each central body of the device is bonded to a body of gripping and single pressure 3 for the whole of the device. This body of gripping and pressure 3 will be used to ensure a catch in hand and an effective and easy handling of the device.

The lower face 4 of the central body 1 shows several characteristics which are represented on FIG. 2: the lower face 4 presents an external cutting which creates, on the external circumference of the central body 1, an acute edge 5. This edge 5 is intended to come into contact with the patient's skin and must be acute enough to leave a print on the skin when the doctor or a nurse applies a pressure to the body of gripping and pressure 3. Preferably, this edge 5 presents an angle ranging between 70° and 80° with the vertical plane of the external circumference of the central body 1, so that the angle of the edge is acute. Thus, this edge 5 will at the same time makes it possible to stop the descent of needle 8 and to leave a print on the arm of the patient, this print encircling the points of penetration of the allergen composition contained in the kit of the invention.

Advantageously, the lower face 4 presents in its center a compartment 20, which carries the multi-points needle 8. This compartment is bonded to the lower face 4 and the wall of this compartment extends vertically to the bottom perpendicularly with the lower face 4. In this provision, compartment 20 has the same vertical central axis as that of the central body. This mode of realization is preferred for an effective penetration of needle 8 in the skin of the patient.

Preferably, the height of compartment 20 is such as the lower end of compartment 20 exceeds by few millimeters the horizontal plane of the end of edge 5. Compartment 20 defines a cavity 21.

According to a second mode of realization represented FIG. 3, cavity 21 is prolonged inside the central body 1. In this mode of realization, the central body 1 is not full: its interior is dug with regard to compartment 20.

According to a mode of realization represented FIG. 4, compartment 20 and cavity 21 can be covered with any material membrane or film or can contain a container, and form a little cell 6. Preferably, the hollow little cell 6 is of cylindrical form and its height lies between 0.2 centimeter and 1 centimeter.

Advantageously, cavity 21 of compartment 20 and/or the central body 1, or little cell 6, contains whole or part of the allergen composition to be inoculated to the patient.

Compartment 20, or if necessary little cell 6, has or carries at its lower end 7 a multi-points needle 8, which is intended to perforate the skin when a pressure is exerted by the doctor or the nurse on the body of pressure 3 and to allow the penetration of the allergen composition.

FIG. 5 represents a multi-points needle 8 which includes at least a crown 12 on which points 13 are fixed or positioned. The fact that the needle is laid out in crown is preferable for a correct penetration of the allergen composition.

According to a particular mode of realization, needle 8 has several concentric crowns 12, in order to increase the number of points while maintaining a restricted diameter for the needle, and in order to improve the penetration of the allergen composition in the patient's skin. According to a preferred mode of realization of the invention, the multi-points needle 8 contains eight points 13 equally distributed, the aforementioned 8 points 13 being distributed on one or two crowns. Preferably, the multi-points needle 8 presents two concentric crowns with a diameter ranging from 1.5 millimeter to 3.5 millimeter, each crown presenting 4 points 13 with a length ranging from 1 to 3 millimeters, angularly positioned in alternation.

The central body is bonded on all its circumference with a flexible ring 16. This flexible ring has the same external format as the central body, so as to be fixed on all its circumference or its perimeter on the external circumference of the central body 1. Thus, the flexible ring 16 allows a controlled vertical translation of the central body 1 at the time of the application of a vertical pressure on the body of pressure 3. The flexible ring 16 consists of materials enabling it to be flexible and resilient, in order to accompany and to limit the descent of the central body 1 when a pressure is made on the body of pressure and gripping 3. This flexible ring 16 must have a sufficient width to entirely accompany the descent of the central body 1 when a pressure is made on body 3. However, this width should not be too important, so that the resistance of material constituting the flexible ring 16 takes part in stopping the descent of the central body 1 and consequently, in stopping the penetration of the multi-points needle 8 in the arm of the patient. The space located under faces 18 and 19 of the flexible ring and the central body 1, is hereafter indicated space 14. According to a preferred mode of realization of the invention, the allergen composition to be inoculated is not only in compartment 20 or cavity 21 or little cell 6 but also in space 14.

The outer edge of flexible ring 16 is bonded on all its circumference or its perimeter to a rigid support 17. This rigid support 17 has for role to position and maintain the kit onto the arm of the patient. Its inner edge is of the same form than the outer edge of flexible ring 16. The anchoring point of the outer edge of flexible ring 16 to the rigid support 17 is strong enough, so that the flexible ring 16 can accept the pressure applied via the body of pressure and gripping 3 without disuniting from rigid support 17.

The cutaneous kit according to the invention includes a blister 15 fixed on the lower face of support 17 and covering the totality of the lower surface of the kit. This blister 15 makes it possible to close spaces 14, and compartments 20 or little cells 6, containing the allergen(s) composition(s). This blister is removable to allow the realization of the test.

A preferred mode of realization of the invention provides that the cutaneous diagnostic kit for atopy can comprise three central bodies 1. Each central body 1 can contain in its cavity 21 of its compartment 20 and/or its central body 1, or in its little cell 6, as in its space 14, an allergen composition to be inoculated. This allergen composition can be different from one central body to another, on the same kit. Thus, the kit according to the invention can carry as much different allergens compositions than it has central bodies. In this way, in only one application of the device according to the invention, the doctor will be able to determine various patients' allergies. On the same kit, the central bodies are one of the other separated, by variable distances. According to a particular mode of realization of the invention represented on FIG. 6, distance 9 separating the first and the second central body is different from distance 10 separating the second and the third central body, each one of the aforesaid distances 9 and 10 being different from distance 11 separating the first and the third central body. The difference in value between the distances separating the various central bodies will allow an easier identification at the time of the reading of the test of the allergens compositions applied by each central body. According to a particular mode of realization of the invention, distance 9 ranges between 1 centimeter and 3 centimeters, distance 10 lies between 2 centimeters and 4 centimeters and distance 11 lies between 3 centimeters and 7 centimeters. In this way, distances 9, 10 and 11 are sufficiently important to avoid overlaps between the reactions of the various central bodies, as well as errors of interpreting reactions between the allergen's composition and the skin.

The cutaneous kit according to the invention can be carried out of only one casting.

The cutaneous kit according to the invention includes at least an allergen composition, located in cavity 21 of compartment 20 and/or the central body 1, or in little cell 6, as in existing space 14 between the blister 15 and the lower faces 18 and 19 of the flexible ring 16 and of the central body 1. Thus, the pressure which is exerted on the body and gripping 3 has two consequences: on one hand, the multi-points needle 8 will penetrate the patient's skin, allowing an intradermal presentation of the allergen composition. On the other hand, the allergen composition being also in space (14), the pressure on the body of pressure 3 will allow an epidermic presentation of the allergen composition, this presentation on the patient's skin being done within the limits of the circle defined by the edge 5 in contact with the skin.

The allergen composition contained in the central body is preferably in a gel form, which prevents that it runs out of space (14) when the blister (15) is removed. However, it can be also presented in liquid form, emulsion form or suspension form.

According to a particular mode of realization of the invention, the only one of several allergen(s) composition(s) contained in the kit of the invention include each one a mixture of allergens, each mixture being made up of allergens belonging to a precise class of allergens responsible for the most frequent allergies. The mixture consists of various allergens of its class in equal proportions. The volume of a mixture is thus from 0.5 microliter to 2 microliters, and the concentration of the allergens is 100 to 300 IR/ml with a preference of 300 IR when technically possible.

According to a mode of realization of the invention, a mixture corresponding to a central body is made up of pollens' allergens, which can contain pollens of graminaceous, herbaceous and trees' pollens. This mixture of pollens' allergens can contain a high peptide concentration of proteins PR10, pectase lyase and profilines, natural or synthetic These proteins are the fundamental major proteins of the class of pollens. The allergen composition can besides include a mixture of food allergens, which can contain a high peptide concentration of chitinases, Lipids Proteins Transfer, natural or synthetic. Lastly, the allergen composition can include a mixture of environmental domestic allergens, being able to be made up of acarids' allergens, of superficial body growths of dog and cat, as well as moulds. This mixture of environmental domestic allergens can include a high peptide concentration of tropomyosines, profilines and cystine proteases, natural or synthetic.

However, a preferred realization of the invention expects that the mixture of allergens comprises, in equal proportion, the various allergens or their fundamental major proteins of each three already quoted classes: class of pollens, class of food allergens, class of environmental domestic allergens.

1. Cutaneous diagnostic kit for patient's atopy applied by a doctor or a nurse, characterized in that it includes:
at least a central body of cylindrical external form (1) of which the upper face (2) is plane and bonded to a single body of gripping and pressure (3) and whose lower face (4) presents a form making on the external circumference of the aforesaid central body an acute edge (5), the aforementioned central body including at least a cavity (21) or hollow little cell (6) coaxial of the aforesaid central body (1), the aforementioned cavity (21) or little cell (6) being part or bonded to the aforesaid central body (1) and being of such a height that its lower end (7) goes beyond the horizontal plane of the acute end of the edge (5) and supports a multi-points needle (8),
The aforementioned central body (1) being bonded on all its circumference with the inner edge of a flexible ring (16), the outer edge of the aforementioned flexible ring (16) being bonded on all its circumference to a rigid support (17),
A blister (15) fixed on the lower face of the support (17) and covering the totality of the lower surface of the kit,
At least an allergen composition, located in the cavity (21) or the little cell (6), as well as in space (14) existing between the blister (15) and lower faces (18 and 19) of the flexible ring (16) and the central body (1).

2. Cutaneous diagnostic kit for patient's atopy applied by a doctor or a nurse, according to the claim 1, characterized in that it comprises three central bodies (1).

3. Cutaneous diagnostic kit for patient's atopy applied by a doctor or a nurse, according to claim 2, characterized in that the distance (9) separating the first and the second central body is different from the distance (10) separating the second and the third central body, each one of the aforesaid distances (9) and (10) being different from the distance (11) separating the first and the third central body.

4. Cutaneous diagnostic kit for patient's atopy applied by a doctor or a nurse, according to claim 3, characterized in that the distance (9) lies between 1 centimeter and 3 centimeters, in that the distance (10) lies between 2 centimeters and 4 centimeters and in that the distance (11) lies between 3 and 7 centimeters.

5. Cutaneous diagnostic kit for patient's atopy applied by a doctor or a nurse, according to claim 1, characterized in that the cavity (21) or the little cell (6) has a height ranging between 0.2 centimeter and 1 centimeter.

6. Cutaneous diagnostic kit for patient's atopy applied by a doctor or a nurse, according to any of claims 1 to 5, characterized in that the aforementioned multi-points needle (8) presents at least a crown (12) on which are positioned or fixed the points (13).

7. Cutaneous diagnostic kit for patient's atopy applied by a doctor or a nurse, according to any of claims 1 to 6, characterized in that it presents several concentric crowns (12).

8. Cutaneous diagnostic kit for patient's atopy applied by a doctor or a nurse, according to any of claims 1 to 7, characterized in that the aforementioned multi-points needle (8) contains 8 points (13) equally distributed, the aforementioned 8 points (13) being distributed on one or two crowns.

9. Cutaneous diagnostic kit for patient's atopy applied by a doctor or a nurse, according to any of claims 6 to 8, characterized in that the aforementioned multi-points needle (8) presents two concentric crowns, each crown presenting 4 points angularly positioned in alternation.

10. Cutaneous diagnostic kit for patient's atopy applied by a doctor or a nurse, according to one of claims 1 to 9, characterized in that the aforementioned allergen composition can be in the form of gel, of liquid, emulsion or suspension.

11. Cutaneous diagnostic kit for patient's atopy applied by a doctor or a nurse, according to any of claims 1 to 10, characterized in that the aforementioned allergen composition includes a mixture of pollens' allergens.

12. Cutaneous diagnostic kit for patient's atopy applied by a doctor or a nurse, according to claim 11, characterized in that the aforementioned mixture of allergens of pollens includes pollens of graminaceous, herbaceous and trees' pollens.

13. Cutaneous diagnostic kit for patient's atopy applied by a doctor or a nurse, according to any of the claims 11 or 12, characterized in that the aforementioned mixture of pollens' allergens includes a high peptide concentration of proteins PR10, pectase lyase and profilines, natural or synthetic.

14. Cutaneous diagnostic kit for patient's atopy applied by a doctor or a nurse, according to any of claims 1 to 10, characterized in that the aforementioned allergen composition includes a mixture of food allergens.

15. Cutaneous diagnostic kit for patient's atopy applied by a doctor or a nurse, according to the claim 14, characterized in that the aforementioned mixture of food allergens includes a high peptide concentration of chitinases, Lipids Proteins Transfer, natural or synthetic.

16. Cutaneous diagnostic kit for patient's atopy applied by a doctor or a nurse, according to any of claims 1 to 10, characterized in that the aforementioned allergen composition includes a mixture of domestic environmental allergens.

17. Cutaneous diagnostic kit for patient's atopy applied by a doctor or a nurse, according to claim 16, characterized in that the mixture of domestic environmental allergens includes the acarids' allergens, of superficial body growths of dog and cat, as well as of moulds.

18. Cutaneous diagnostic kit for patient's atopy applied by a doctor or a nurse, according to any of the claims 16 or 17 characterized in that the aforementioned mixture of domestic environmental allergens includes a high peptide concentration of tropomyosines, of profilines and cystine proteases, natural or synthetic.

19. Cutaneous diagnostic kit for patient's atopy applied by a doctor or a nurse, according to any of claims 1 to 18, characterized in that each one of the aforesaid central bodies (1) contains in its space (14) an allergen composition different from that of the other central bodies (1).

20. Cutaneous diagnostic kit for patient's atopy applied by a doctor or a nurse, according to any of claims 1 to 19, characterized in that it consists of materials of single use.
